SEQUENCE LISTING (2 sequences)

SEQ ID NO: 1 - DNA, 19 bp, Artificial Sequence
  Pkd1 primer
gaatatcggtgggagatat

SEQ ID NO: 2 - DNA, 21 bp, Artificial Sequence
  Pkd2 primer
gcatcttgacctacggcatga

(19) United States
(12) Patent Application Publication
(10) Pub. No.: US 2023/0009145 A1
(43) Pub. Date: Jan. 12, 2023

(54) INHIBITION OF TMEM16A BY BENZBROMARONE OR NICLOSAMIDE FOR TREATING POLYCYSTIC KIDNEY DISEASE AND/OR POLYCYSTIC LIVER DISEASE

(71) Applicants: UNIVERSITÄT REGENSBURG, REGENSBURG (DE); FRIEDRICH-ALEXANDER-UNIVERSITÄT ERLANGEN-NÜRNBERG, ERLANGEN (DE)

(72) Inventors: KARL KUNZELMANN, REGENSBURG (DE); RAINER SCHREIBER, REGENSBURG (DE); BJÖRN BUCHHOLZ, HESSDORF (DE)

(21) Appl. No.: 17/782,643

(22) PCT Filed: Dec. 9, 2020
(86) PCT No.: PCT/EP2020/085338

§ 371 Date: Jun. 4, 2022

(30) Foreign Application Priority Data
Dec. 18, 2019 (EP) 19 217 482.9

(51) Int. Cl. A61K 31/343; A61K 31/167; A61K 31/55; A61K 38/31; A61K 31/436; A61P 13/12; A61P 1/16; A61K 31/426; A61K 45/06

(52) U.S. Cl. CPC A61K 31/343; A61K 31/167; A61K 31/55; A61K 38/31; A61K 31/436; A61P 13/12; A61P 1/16; A61K 31/426; A61K 45/06

(57) ABSTRACT

The present invention relates to a compound for use in a method of treating a pathological condition selected from polycystic kidney disease, polycystic liver disease, and a combination thereof. The present invention further relates to a composition for use in a method of treating a pathological condition selected from polycystic kidney disease, polycystic liver disease, and a combination thereof.

### FIELD OF THE INVENTION

The present invention relates to a compound for use in a method of treating a pathological condition selected from polycystic kidney disease, polycystic liver disease, and a combination thereof. The present invention further relates to a composition for use in a method of treating a pathological condition selected from polycystic kidney disease, polycystic liver disease, and a combination thereof.

### BACKGROUND OF THE INVENTION

Polycystic kidney diseases (PKDs) comprise a number of inherited disorders that lead to bilateral renal cyst development. The hereditary forms of polycystic kidney disease include a wide range of heterogeneous diseases of great clinical importance, of which autosomal dominant PKD (ADPKD) and autosomal recessive PKD (ARPKD) are the main forms. The main hereditary form of polycystic kidney disease (ADPKD) is associated with polycystic liver disease (PLD). Polycystic liver disease may also occur as a distinct genetic disease in the absence of renal cysts but may also lead to renal cysts. Polycystic kidney/liver diseases represent a very significant medical problem.

About 5% of patients requiring renal replacement therapy suffer from ADPKD. Polycystic kidney disease leads to continuous decline of renal function by growth of renal cysts. The dominant form of PKD, ADPKD, has a prevalence of 3.9/10000 and is the predominant cause of PKD for terminal kidney insufficiency in the European Union. ADPKD is characterized by continuous cyst enlargement over time, leading to compression of adjacent healthy parenchyma. In progressed stages of polycystic kidney diseases, the presence of cysts may result in kidney insufficiency, and dialysis and/or kidney transplantation may become necessary.

ADPKD is caused by mutations in PKD1 (polycystin 1) or PKD2 (polycystin 2), but the underlying complex molecular events leading to continuous cyst growth are still poorly understood. In normal renal epithelial cells, PKD1 and PKD2 are located in the so-called primary cilium, where they form a complex of receptor and Ca²⁺ influx channel. Ca²⁺ ions are more concentrated within the primary cilium compared to the cytoplasm, however, Ca²⁺ signals generated within the cilium may occur independent of cytoplasmic Ca²⁺ signaling. Loss of the primary cilium or loss of function of PKD1 or PKD2 leads to relocalization of the polycystins to plasma membrane and endoplasmic reticulum, resulting in disturbed intracellular Ca²⁺ signaling.

The standard therapy for early stages of PKD is usually symptomatic and comprises dietary approaches and treatment of co-occurring hypertension, urinary tract infections, antibiotic treatment, and pain therapy. Presently, about 50% of ADPKD patients need to undergo a dialysis treatment, and the need for a dialysis treatment is associated with reduced life expectancy. Inhibiting cyst growth, thereby preventing and/or delaying the need for a dialysis, may be very beneficial in the treatment of PKD patients. A vasopressin-antagonist, namely Tolvaptan, has been shown to reduce cyst growth and has obtained marketing approval. Furthermore, octreotide, a somatostatin analogue, as well as sirolimus and everolimus, which are mTOR antagonists, have been or are currently tested in clinical studies. However, presently only dialysis and kidney transplantations are available for treating for progressed stages of PKD. Therefore, therapeutic options for treating PKD, particularly progressed stages of PKD, are needed. Furthermore, a means for preventing and/or delaying a patient's need for a dialysis would be highly advantageous.

Schreiber et al. [3] state that TMEM16A plays a role in PKD and that idebenone may inhibit TMEM16A in PKD.

Buchholz et al. [4] relate to inhibiting cyst growth and cyst enlargement using two inhibitors of anoctamin ion channels, namely tannic acid and a more selective inhibitor of anoctamin 1 (TMEM16A).

Benzbromarone was developed in the 1970s as a uricosuric agent and non-competitive inhibitor of xanthine oxidase, to be used in the treatment of gout, especially when allopurinol, a first-line treatment, fails or produces intolerable adverse effects. Benzbromarone is highly effective and well tolerated. Clinical trials as early as in 1981 and recent studies have suggested that it is superior to both allopurinol, a non-uricosuric xanthine oxidase inhibitor, and probenecid, another uricosuric drug. Benzbromarone is a very potent inhibitor of CYP450. Benzbromarone has been shown to potently inhibit TMEM16A [1, 2].

Niclosamide is a derivative of salicylic acid and aniline, which are linked together as an amide (salicylanilide). Niclosamide was introduced in 1959 as a molluscicide and antihelminthic agent. In the form of a salt with 2-aminoethanol (niclosamide ethanolamine) it is used under the names Clonitralid® and Bayluscid® to control water snails that transmit schistosomiasis, as well as for systemic use in humans. Niclosamide has been identified as a potent inhibitor of TMEM16A [2].

The aim of the present invention is to provide a means that is effective in the prevention and/or treatment of PKD and/or PLD, particularly by inhibiting cyst growth. It is a further aim of the invention to provide a compound that effectively inhibits cyst growth in vivo. Particularly, it is an aim of the present invention to prevent cyst growth in order to prevent the need for renal replacement therapy. It is also an aim of the present invention to relief pain of PKD patients by inhibiting cyst growth, since cyst growth often leads to kidney pain. The present invention also aims at preventing cyst rupture, inflammation, and hemorrhage by preventing cyst enlargement. A further aim of the present invention is preventing nephrectomy which may become necessary due to kidneys compressing surrounding organs. Furthermore, the present invention aims at providing a means for preventing and/or delaying a patient's need for a dialysis.

### SUMMARY OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the present invention relates to a compound for use in a method of treating a pathological condition selected from polycystic kidney disease, polycystic liver disease, and a combination thereof, wherein said compound is a TMEM16 inhibitor selected from benzbromarone, niclosamide, and pharmaceutically acceptable salts thereof.

In one embodiment, said pathological condition is a combination of polycystic kidney disease and polycystic liver disease.

In one embodiment, said pathological condition is characterized by cyst development.

In one embodiment, said pathological condition is characterized by increased TMEM16A expression and/or increased TMEM16F expression, preferably is characterized by increased TMEM16A expression in kidney cells.

In one embodiment, said polycystic kidney disease is autosomal dominant polycystic kidney disease (ADPKD) or autosomal recessive polycystic kidney disease (ARPKD), preferably is ADPKD.

In one embodiment, said compound is capable of inhibiting renal cyst growth and/or hepatic cyst growth by inhibiting TMEM16A and/or TMEM16F.

In one embodiment, said compound is administered in an amount of from 10 mg per day to 800 mg per day, preferably 40 mg to 600 mg per day.

In one embodiment, said compound is administered once every 4-8 h, once daily, or once weekly, preferably once daily.

In one embodiment, said compound is administered to a patient in need thereof, wherein said patient is a mammal, preferably a human.

In one embodiment, said compound is administered topically or systemically.

In one embodiment, said compound is administered intravenously, intravascularly, orally, intraarticularly, nasally, mucosally, intrabronchially, intrapulmonarily, intrarenally, intrahepatically, intradermally, subcutaneously, intramuscularly, intraocularly, intrathecally, or intranodally, wherein said compound is preferably administered orally.

In one embodiment, said compound is co-administered with an agent selected from an antihypertensive agent, an antiinfective agent, an antibiotic agent, an analgesic agent, a vasopressin antagonist such as tolvaptan, a somatostatin analogue such as octreotide, and an mTOR antagonist such as sirolimus or everolimus.

In one embodiment, said compound is a biologically active derivative of benzbromarone or a biologically active derivative of niclosamide.

In a further aspect, the present invention relates to a composition for use in a method of treating a pathological condition selected from polycystic kidney disease, polycystic liver disease, and a combination thereof, wherein said composition comprises a compound as defined in any of the embodiments above, and a pharmaceutically acceptable excipient.

In one embodiment, said composition further comprises any of an antihypertensive agent, an antiinfective agent, an antibiotic agent, an analgesic agent, a vasopressin antagonist such as tolvaptan, a somatostatin analogue such as octreotide, an mTOR antagonist such as sirolimus or everolimus, a disintegrant, and a pharmaceutically acceptable carrier.

In this aspect, said pathological condition, said polycystic kidney disease, said polycystic liver disease, and said compound are as defined above.

In a further aspect, the present invention relates to a method of preventing and/or treating a pathological condition selected from polycystic kidney disease, polycystic liver disease, and a combination thereof, wherein said method comprises administering a compound which is a TMEM16A inhibitor selected from benzbromarone, niclosamide, and pharmaceutically acceptable salts thereof to a patient in need thereof.

In this aspect, said pathological condition, said polycystic kidney disease, said polycystic liver disease, and said compound are as defined above.

In a further aspect, the present invention relates to a use of a compound which is a TMEM16A inhibitor selected from benzbromarone, niclosamide, and pharmaceutically acceptable salts thereof for the manufacture of a medicament for preventing and/or treating a pathological condition selected from polycystic kidney disease, polycystic liver disease, and a combination thereof.

In this aspect, said pathological condition, said polycystic kidney disease, said polycystic liver disease, said treating, and said compound are as defined above.

### DETAILED DESCRIPTION

Polycystic kidney diseases (PKDs) comprise a number of inherited disorders that lead to bilateral renal cyst development. Hereditary forms of polycystic liver disease are associated with polycystic kidney disease. These diseases are frequent and represent a very significant medical problem. Enhanced cell proliferation, enhanced apoptosis, and transepithelial chloride secretion are the main causes for cyst expansion. The present inventors herein demonstrate the pro-proliferative role of the Ca²⁺-activated Cl⁻ channel TMEM16A and its essential contribution to fluid secretion into the cyst lumen. Particularly, the present inventors herein show, firstly, that the compounds benzbromarone and niclosamide inhibit renal cyst growth ex vivo and in vitro, secondly, that a knockout of TMEM16A inhibits cyst formation in vivo, and, thirdly, that benzbromarone inhibits cyst formation in vivo.

The term “polycystic kidney disease” or “PKD”, as used herein, relates to a pathological condition in which the renal tubules become structurally abnormal which results in the development and growth of multiple cysts within the kidney. In one embodiment, PKD relates to a genetic disorder, such as autosomal dominant polycystic kidney disease (ADPKD) or autosomal recessive polycystic kidney disease (ARPKD). Genetic mutations of ADPKD patients in any of the two known genes PKD1 and PKD2, and a potential, presently unknown gene PKD3 have similar phenotypical presentations. Gene PKD1 encodes a protein, polycystin-1, involved in regulation of cell cycle and intracellular calcium transport in epithelial cells, and is responsible for 85% of the cases of ADPKD. PKD2 encodes polycystin-2 which is also called TRPP2 since sequence homology has placed polycystin-2 into the family of transient receptor potential (TRP) cation channels. ARPKD is less common than ADPKD. PKD may be accompanied by development of cysts in the liver, i.e. by polycystic liver disease. In one embodiment, a polycystic kidney disease may further relate to a pathological condition selected from nephronophthisis, Meckel syndrome, Bardet-Biedl syndrome, Joubert syndrome, oral-facial-digital syndrome, glomerulocystic kidney disease, tuberous sclerosis complex, autosomal dominant tubulointerstitial kidney disease, von Hippel-Lindau disease, and medullary sponge kidney.

In one embodiment, knockdown of PKD1 or PKD2 enhances expression of TMEM16A, increases basal intracellular Ca²⁺levels and augments purinergic/inositol trisphosphate (IP3) induced Ca²⁺release from endoplasmic reticulum. In one embodiment, ryanodine receptors are not expressed in renal epithelial cells and caffeine has no effects on intracellular Ca²⁺ concentrations. In one embodiment, intracellular Ca²⁺ signals in primary mouse epithelial cells, mouse M1 collecting duct cells, and MDCK cells are largely reduced by knockdown or blockade of TMEM16A, and TMEM16A is a major pathogenic factor for enhanced Ca²⁺ release from IP₃-sensitive Ca²⁺ stores in autosomal dominant polycystic kidney disease (ADPKD).

The term “polycystic liver disease” or “PLD”, as used herein, relates to a pathological condition in which cysts grow throughout the liver. PLD occurs either in isolation, in which patients have cysts only in the liver, or in combination with polycystic kidney disease, in which patients have cysts in both the liver and the kidney. PLD is most common in patients who also suffer from polycystic kidney disease. In one embodiment, PLD relates to ADPLD which is autosomal dominant polycystic liver disease. In one embodiment, a patient suffering from PKD, preferably ADPKD, further suffers from PLD.

The term “TMEM16”, as used herein, relates to proteins which are also known as anoctamins, and which are involved in the variety of functions including ion transport and regulation of other membrane proteins. TMEM16 proteins are a family of proteins comprising TMEM16A, TMEM16B, TMEM16C, TMEM16D, TMEM16E, TMEM16F, TMEM16G, TMEM16H, TMEM16J, and TMEM16K. TMEM16A and TMEM16B function as Ca²⁺-activated Cl⁻ channels. In one embodiment, TMEM16 preferably relates to TMEM16A and/or TMEM16F. In one embodiment, TMEM16, preferably TMEM16A and/or TMEM16F, controls intracellular Ca²⁺ signals. In one embodiment, TMEM16A and TMEM16F increase intracellular Ca²⁺levels close to the plasma membrane, wherein membrane-near Ca²⁺ activates CFTR and membrane exocytosis. In one embodiment, TMEM16A/F is an ideal therapeutic target in PKD and/or PLD, since upregulation of TMEM16A/F in PKD/PLD i) augments fluid secretion, ii) increases proliferation, and iii) induces cellular apoptosis. In one embodiment, the expression of the ion channel TMEM16A is increased in kidney cells of patients having PKD compared to kidney cells of healthy controls. In one embodiment, TMEM16A leads to increased proliferation of cyst epithelium in vitro, ex vivo in cyst kidneys, and/or in vivo in a PKD1-KO mouse model. In one embodiment, TMEM16F leads to proliferation of cyst epithelium in vitro in a plMDCK cyst model.

The term “inhibitor”, as used herein, relates to a compound that inhibits a target, such as a TMEM16 protein. In one embodiment, said inhibitor is an inhibitor of TMEM16A and/or TMEM16F. In one embodiment, said inhibitor is a specific inhibitor which exclusively binds to and inhibits one target, such as TMEM16A or TMEM16F. In one embodiment, said inhibitor may also have an effect on more than one target, i.e. an inhibitor may have an effect on different TMEM16 proteins, such as an effect on both TMEM16A and TMEM16F. In one embodiment, said TMEM16 inhibitor, preferably TMEM16A and/or TMEM16F inhibitor, is selected from the group consisting of niclosamide, benzbromarone, and pharmaceutically acceptable salts thereof. In one embodiment, inhibiting the ion channel TMEM16A using compounds benzbromarone and/or niclosamide results in the inhibition of cyst growth and/or cyst development. In one embodiment, the terms cyst growth and cyst development are used interchangeably. In one embodiment, benzbromarone and niclosamide are more specific and effective at lower concentrations than idebenone in inhibiting TMEM16A. In one embodiment, idebenone inhibits ROS and thus affects various signaling pathways other than a TMEM16A-related pathway. In one embodiment, tannic acid is an unspecific inhibitor and inhibits TMEM16A and many other ion channels. In one embodiment, CaCCinh-A01 is an inhibitor that is disadvantageous for in vivo use, for example since it is not orally available.

The term “benzbromarone”, as used herein, relates to a uricosuric agent and is also referred to as (3,5-dibromo-4-hydroxyphenyl)(2-ethyl-1-benzofuran-3-yemethanone. The term “benzbromarone” further relates to salts, particularly pharmaceutically acceptable salts, of benzbromarone. In one embodiment, benzbromarone blocks renal cyst growth in vivo, such as in vivo in inducible tubule-specific PKD1 knockout (PKD1−/−) mice. The term “benzbromarone” further relates to biologically active derivatives of benzbromarone which have the same effect on TMEM16A/F as benzbromarone. In one embodiment, a biologically active derivative of benzbromarone has the same therapeutic effect as benzbromarone, preferably the same therapeutic effect on a pathological condition selected from polycystic kidney disease, polycystic liver disease, and a combination thereof, as benzbromarone. In one embodiment, a biologically active derivative of benzbromarone has the same inhibiting effect on cyst growth as benzbromarone. In a preferred embodiment, a derivative of benzbromarone, preferably biologically active derivative of benzbromarone, has the same effect on TMEM16A/F as benzbromarone, and the same therapeutic effect as benzbromarone and/or the same inhibiting effect on cyst growth as benzbromarone.

The term “niclosamide”, as used herein, refers to a drug which is commonly used to treat tapeworm infestations. It also referred to as 5-Chlor-N-(2-chlor-4-nitrophenyl)-2-hydroxybenzamid having a formula C₁₃H₈Cl₂N₂O₄. The term “niclosamide” further relates to salts, particularly pharmaceutically acceptable salts, of niclosamide, such as niclosamide-ethanolamine and/or niclosamide-olamine. The term “niclosamide-ethanolamine”, as used herein, refers to an ethanolamine salt of niclosamide which is an antihelminthic compound. The term “niclosamide-olamine”, as used herein, refers to clonitralid which is a niclosamide ethanolamine salt having a formula C₁₃H₈Cl₂N₂O₄ C₂H₇NO. In one embodiment, niclosamide blocks renal cyst growth in vivo, such as in vivo in PKD1−/− mice. The term “niclosamide” further relates to biologically active derivatives of niclosamide which have the same effect on TMEM16A/F as niclosamide. In one embodiment, a biologically active derivative of niclosamide has the same therapeutic effect as niclosamide, preferably the same therapeutic effect on a pathological condition selected from polycystic kidney disease, polycystic liver disease, and a combination thereof, as niclosamide. In one embodiment, a biologically active derivative of niclosamide has the same inhibiting effect on cyst growth as niclosamide. In a preferred embodiment, a derivative of niclosamide, preferably biologically active derivative of niclosamide, has the same effect on TMEM16A/F as niclosamide, and the same therapeutic effect as niclosamide and/or the same inhibiting effect on cyst growth as niclosamide. In one embodiment, nitazoxanide is an exemplary biologically active derivative of niclosamide.

In one embodiment, “biologically active” in the context of a derivative of benzbromarone or niclosamide means that the derivative has the same or a similar effect on TMEM16A/F as benzbromarone and niclosamide, respectively. In one embodiment, “biologically active” in the context of derivatives of benzbromarone or niclosamide means that the derivatives have the same or a similar effect on a pathological condition selected from polycystic kidney disease, polycystic liver disease, and a combination thereof, as benzbromarone and niclosamide, respectively. In one embodiment, the term “biologically active” in the context of a derivative of benzbromarone or niclosamide means that the derivative is capable of eliciting a therapeutic response, preferably the same therapeutic response as benzbromarone and niclosamide, respectively. In one embodiment, the term “derivative” refers to “biologically active derivative”. In one embodiment, a derivative of benzbromarone or niclosamide is advantageous in that it has an enhanced bioavailability, such as enhanced oral bioavailability, and/or has an enhanced tolerability, compared to benzbromarone or niclosamide, respectively.

The term “cyst development”, as used herein, relates to abnormal growth and/or formation of cysts, which typically are fluid-filled, in a pathological condition such as polycystic kidney disease. Diseases such as polycystic kidney disease result in the development and growth of multiple cysts within the body, such as within the kidney. In PKD, the abnormal gene often exists in all cells of the body and thus cysts may also occur in other tissues such as the liver, seminal vesicles, and pancreas. In one embodiment, cyst growth is reduced in mice with a double knockout of the genes PKD1 and TMEM16A. In one embodiment, cyst growth in a PKD patient, preferably an ADPKD patient, is inhibited by a TMEM16A inhibitor, preferably selected from benzbromarone and niclosamide. In one embodiment, cyst development in the kidney of a PKD patient occurs in the entire tubule system, i.e. in the nephron and the collecting duct of the kidney, preferably involving the cells of the collecting duct of the kidney. In one embodiment, cyst development in the liver involves epithelial cells of the biliary tract. In one embodiment, TMEM16A is expressed in the entire tubule system, i.e. in the nephron and the collecting duct of the kidney, and a TMEM16A inhibitor is thus advantageous over tolvaptan, since tolvaptan only has an effect on the collecting duct of the kidney instead of on both the nephron and the collecting duct of the kidney.

The term “capable of inhibiting renal cyst growth and/or hepatic cyst growth”, as used herein, relates to the capability of a compound and/or a composition of the present invention to inhibit the growth of cysts in renal tissue and/or hepatic tissue. Thereby, the growth of cysts is prevented and/or inhibited. In one embodiment, the term relates to the capability of reducing the amount and/or size of cysts, and/or relates to inhibiting growth of cysts. In one embodiment, by inhibiting cyst growth, a compound for use of the present invention allows for preventing or postponing the need for dialysis, and/or allows for preventing nephrectomy, and/or allows for reducing the risk of a PKD patient to acquire a kidney carcinoma. In one embodiment, enhanced proliferation, enhanced cell death and transepithelial chloride secretion through cystic fibrosis transmembrane conductance regulator (CFTR) Cl⁻ channels are the main cause for expansion of the cysts. In one embodiment, chloride secretion is followed by water transport resulting in cyst growth. In one embodiment, a TMEM16A inhibitor inhibits Ca²⁺-activated chloride secretion which plays a role in cyst growth. In one embodiment, a TMEM16A inhibitor further inhibits cAMP-(CFTR−) dependent chloride secretion.

The term “increased expression” or “overexpression”, as used herein, refers to an elevated expression level of as compared to the expression level in a healthy cell and/or a healthy tissue.

The terms “administered” and “administration”, as used herein, relate to applying a therapeutically active agent, particularly a compound for use according to the present invention, to a patient in need thereof. In one embodiment, a compound for use of the present invention is administered topically or systemically. In one embodiment, a compound for use of the present invention is administered intravenously, intravascularly, orally, intraarticularly, nasally, mucosally, intrabronchially, intrapulmonarily, intrarenally, intrahepatically, intradermally, subcutaneously, intramuscularly, intraocularly, intrathecally, or intranodally, preferably orally. In one embodiment, said compound for use is administered intravenously or subcutaneously every 2-4 weeks. In one embodiment, a compound for use of the present invention is administered once every 4-8 h, once daily, once weekly, or once every 2-4 weeks, preferably once daily. In one embodiment, an effective dose of a compound for use is administered to a patient in need thereof. In one embodiment, a compound for use of the present invention is administered in an amount of from 10 mg per day to 800 mg per day, preferably 40 mg to 600 mg per day. In one embodiment, said compound is benzbromarone and is administered in an amount of from 10 mg per day to 300 mg per day, preferably 40 mg to wo mg per day. In one embodiment, said compound is niclosamide and is administered in an amount of from 100 mg per day to 800 mg per day, preferably 400 mg to 600 mg per day. In one embodiment, a compound for use is administered to a patient during a dialysis session or in between two dialysis sessions.

The terms “effective dose” and “effective amount”, as used herein, refer to a dose of a drug, such as benzbromarone or niclosamide, which is in the range between the dose sufficient to evoke a desired therapeutic effect and the maximum tolerated dose.

The term “patient”, as used herein, relates to a mammal, preferably a human. In one embodiment, said patient suffers from a pathological condition which is polycystic kidney disease and/or polycystic liver disease. In one embodiment, a patient is an ADPKD patient characterized by rapid cyst growth. The term “patient characterized by rapid cyst growth”, as used herein, comprises ADPKD patients with a Mayo classification 1C-1E [5], and/or patients with a loss of the estimated glomerular filtration rate (eGFR)≥5 ml/min/1.73 m² in 1 year or ≥2.5 ml/min/1.73 m²/year within a period of≥5 years, and/or patients presenting with chronic kidney disease (CKD) stage≥2 in the age of 18-39 years or CKD stage≥3 in the age of 40-50 years [6].

The term “co-administered”, as used herein, relates to a combined administration of a compound for use of the present invention with any other therapeutic agent, such as with an agent selected from an antihypertensive agent, an antiinfective agent, an antibiotic agent, an analgesic agent, a vasopressin antagonist such as tolvaptan, a somatostatin analogue such as octreotide, and an mTOR antagonist such as sirolimus or everolimus.

The term “composition”, as used herein, relates to a composition comprising benzbromarone, niclosamide, or a pharmaceutically acceptable salt of benzbromarone or niclosamide, and further comprising any other agent, such as a further therapeutic agent or an excipient. In one embodiment, a composition further comprises any of an antihypertensive agent, an antiinfective agent, an antibiotic agent, an analgesic agent, a vasopressin antagonist such as tolvaptan, a somatostatin analogue such as octreotide, an mTOR antagonist such as sirolimus or everolimus, a disintegrant, and a pharmaceutically acceptable carrier. In one embodiment, a composition is formulated as an oral dosage form.

The term “excipient”, as used herein, relates to a pharmaceutically acceptable substance that is formulated alongside an active ingredient in a composition, wherein the excipient has the purpose of enhancing the properties of the composition, such as long-term stabilization and/or enhancing solubility. For example, an excipient may be a preservative, emulsifier, solubilizer, buffer, or absorption accelerant.

### BRIEF DESCRIPTION OF THE FIGURES

The present invention is now further described by reference to the following figures.

All methods mentioned in the figure descriptions below were carried out as described in detail in the examples.

FIG. 1 shows that TMEM16A augments Ca²⁺ signaling and ion transport in MDCK cells.

A) RT-PCR indicating expression of TMEM16A in MDCK-C7 cells but not in MDCK-M2 cells.

B) Summary of basal Ca²⁺ levels in MDCK-C7 and MDCK-M2 cells.

C,D) ATP or UTP induced peak and plateau Ca²⁺ levels (both 100 μM).

E) Original recordings and summary of ATP or UTP induced transepithelial voltages and effect of TMEM16A-knockout.

F,G) Effect of siRNA on expression of TMEM16A and TMEM16F, respectively, as assessed by semiquantitative RT-PCR.

Mean±SEM (number of cells measured). #significant difference when compared to C7 and scrambled, respectively.

FIG. 2 shows the role of TMEM16A in plasma membrane and primary cilium of MDCK cells.

A) Acetylated tubulin (red/upper panel) and TMEM16A (green/lower panel) in a primary cilium of MDCK cells.

B) Ca²⁺ sensor 5-HT6-G-GECO1 expressed in the primary cilium and near plasma membrane allowing measurement of Ca²⁺ in both compartments.

C,D) Original recordings and summary of Ca²⁺ signals elicited by stimulation with ATP or UTP (both 100 μM) in primary cilium and near plasma membrane.

E,F) Increase of Ca²⁺ in the absence of extracellular Ca²⁺.

G,H) Comparison of purinergic Ca²⁺ increase in MDCK-C7 (expressing TMEM16A) and MDCK-M2 (not expressing TMEM16A). Bars=2 μm.

Mean±SEM (number of cells measured). #significant difference when compared to membrane (p<0.05; unpaired t-test). §significant difference when compared to MDCK-C7 (p<0.05; unpaired t-test).

FIG. 3 shows a M1 renal organoid and cyst model.

A) RT-PCR analysis of mRNA expression of ion channels TMEM16A, TMEM16F, αβγ-ENaC (abg-Scnn1), and PKD1, as well as PKD2, and the receptor patched 1,2 (Ptch1,2). Similar expression patterns were found in mouse kidney and the collecting duct cell line M1. +/− indicate presence/absence of reverse transcriptase.

B) Growth of renal organoid in matrigel within 9 days. Bars=20 μm.

C) Reconstructed 3D image from a renal M1 organoid. Bars=20 μm.

D,E) Differential interference contrast (DIC) image and immunocytochemistry of a cross-section of an organoid. Green/light gray, primary cilia; red/medium gray, CFTR; blue/dark gray, DAPI. Bars=20 μm.

F) M1 organoid (middle) and cystic expansion by shRNA knockdown of PKD1 and PKD2. Bumetanide (100 μM) was continuously present in matrigel, indicating contribution of fluid secretion to cyst development (in shPKD1 and shPKD2 treated cells), which is absent in M1 renal organoids. Bars=50 μm.

G) Increase in volume during 9 days of organoid/cyst growth in matrigel and inhibition of cyst growth by bumetanide.

H) Increase in proliferative activity in shPKD1 and shPKD2 treated cells, as indicated by Ki-67 staining.

Bars=20 vtm. Mean±SEM (number of organoids measured). #significant difference when compared with scrambled (p<0.05; unpaired t-test). §significant difference when compared to absence of bumetanide (p<0.05; unpaired t-test).

FIG. 4 shows increased proliferation by knockdown of PKD1 or PKD2.

A) Ki-67 staining (red/medium gray) indicating increase in proliferation and enhanced expression of TMEM16A (green/light gray) in M1 cysts caused by knockdown of PKD1 or PKD2.

B) Western blot indicating small hairpin (sh) RNA-knockdown of PKD1 and PKD2, respectively.

C,D) Increase in cyst volume and proliferation upon knockdown of PKD1/PKD2, and inhibition by 5 μM benzbromarone or CaCCinhA01.

Bars=20 μm. Mean±SEM (number of organoids measured). #significant difference when compared control (p<0.05; ANOVA). §significant difference when compared to scrambled (p<0.05; ANOVA).

FIG. 5 shows induction of Cl⁻ secretion by knockdown of PKD1 or PKD2.

A,B) Ussing chamber recordings on M1 cells of polarized grown permeable supports (2D culture). Enhanced Cl⁻ secretion by luminal stimulation of ATP (100 μM) or forskolin/IBMX (IF; 2 μM/100 μM) in monolayers lacking expression of PKD1 or PKD2.

C-E) Summaries for calculated basal short circuit currents (I_{sc}) and I_{sc} activated by ATP and forskolin/IBMX, respectively.

Mean±SEM (number of organoids measured). #significant difference when compared control (p<0.05; ANOVA). §significant difference when compared to scrambled (p<0.05; ANOVA).

FIG. 6 shows upregulation of TMEM16A is essential for enhanced Ca²⁺ signaling upon knockdown of PKD1 and PKD2.

A) Western blot indicating siRNA-knockdown of TMEM16A in M1 collecting duct cells.

B-D) Original recordings and summaries of basal Ca²⁺ and ATP (100 μM) induced Ca²⁺ increase (Fura2) in control cells (scrbld), and cells with a knockdown of PKD1 or PKD2, respectively.

E,F) Original recordings and summaries of ATP-induced Ca²⁺ increase in cells lacking expression of TMEM16A (siT16A).

G) Expression of TMEM16A in M1 control cells (scrbld) and cells lacking expression of PKD1 or PKD2.

H-J) Original recordings and summaries of the effect of ATP on ER Ca²⁺ levels in control cells and cells lacking expression of PKD1 or PKD2.

K) Attenuated ATP-induced Ca²⁺ release after knockdown of TMEM16A. Bars=20 μm.

Mean±SEM (number of monolayers measured). #significant difference when compared scrbld (p<0.05; ANOVA). §significant difference when compared to control (p<0.05; ANOVA).

FIG. 7 shows that TMEM16A is essential for enhanced Ca²⁺ store release by knockdown of PKD1 and PKD2.

A,B) Lack of effects of caffeine on intracellular Ca²⁺ and lack of expression of RyR1-3 in mouse primary renal medullary and M1 collecting duct cells.

C,D) CPA (10 μM) induced store release in the presence or absence PKD1/PKD2.

E,F) CPA-induced store release was strongly attenuated by siRNA-knockdown of TMEM16A.

G-J) Original recordings and summaries of CPA-induced Ca²⁺ store release and SOCE in the presence of SK&F96365 and YM58483 (both 5 μM).

Mean±SEM (number of monolayers measured). #significant difference when compared scrbld (p<0.05; ANOVA). §significant difference when compared to absence of siT16A or SK&F96365/YM58483, repectively (p<0.05; ANOVA).

FIG. 8 shows the contribution of TMEM16A to augmented Ca²⁺ signaling in ADPKD.

Proposed model suggesting cellular mislocalization of PKD2 and PKD1 in the ER, and upregulation/mislocalization of TMEM16A, upon knockout of PKD1 and PKD2, respectively. Ca²⁺ increase upon purinergic (P2Y) receptor stimulation is enhanced by knockout of PKD1/PKD2. TMEM16A strongly contributes to enhanced Ca²⁺ signals probably by tethering IP₃R to the plasma membrane and/or by operating as a counter-ion channel to compensate Ca²⁺-diffusion potentials.

FIG. 9 shows the effect of the TMEM16A-blocker benzbromarone on cyst development in ADPKD. The experiments were performed with PKD1−/− mice. Benzbromarone significantly inhibits cyst growth.

FIG. 10 shows the inhibition of pathologic cell proliferation in ADPKD (PKD1−/− mice) by treatment with the TMEM16A—blocker benzbromarone.

FIG. 11 shows that niclosamide and nitazoxanide inhibit cyst growth in a dose-dependent manner. Polycystin-1-deficient collecting duct (plMDCK) cells were resuspended within a collagen I matrix where they spontaneously form cysts and grow in a secretion-dependent manner in the presence of 10 μM forskolin for 5 days. Medium was supplemented with either 0.1 μM or 1 μM niclosamide or 0.1 μM or 1 μM nitazoxanide. Thereafter, cyst volumes were analyzed. A) Mean cyst volumes±SEM (control=set 100%) from three individual experiments comprising the analysis of 310-330 cysts per condition. B) Photos show representative cysts at day 5. * significant compared to control. § significant compared to 0.1 μM niclosamide. # significant compared to 0.1 μM nitazoxanide. It is shown that niclosamide and niclosamide derivative nitazoxanide inhibit cyst growth in vitro.

In the following, reference is made to the examples, which are given to illustrate, not to limit the present invention.

### EXAMPLES

### Example 1

In the following, materials and methods are described that were used for obtaining the results presented in the further examples.

Cells, Virus Production RT-PCR, cDNA:

MDCK M2 and C7 cell lines were cultured in DMEM supplemented with 10% Fetal Bovine Serum (FBS). M1 cells were cultured DMEM/F12 medium supplemented with 5% (v/v) fetal bovine serum (FBS), 1% Insulin-Transferrin-Selenium boox (ITS), and 1% L-Glutamine 200 mM (all from Capricorn Scientific GmbH, Ebsdorfergrund, Germany) at 37° C. in a humidified incubator in 5% (v/v) CO₂. M1 cells were transduced to downregulate Pkd1 and Pkd2. Cells were infected with lentiviral recombinant vectors containing the shRNAs of mouse Pkd1 (5′-GAATATCGGTGGGAGATAT; SEQ ID NO. 1) and Pkd2 (5′-GCATCTTGACCTACGGCATGA, SEQ ID NO. 2) with YFP_{I152L}, as previously described. Stable transfected M1 cells were maintained in the presence of 5 μg/ml of Puromycin (Thermo Fisher Scientific, Darmstadt, Germany).

For semi-quantitative RT-PCR total RNA from M1 cells, MDCK cells and murine kidney were isolated using NucleoSpin RNA II columns (Macherey-Nagel, Duren, Germany). Total RNA (1 μg/50 μl reaction) was reverse-transcribed using random primer (Promega, Mannheim, Germany) and M-MLV Reverse Transcriptase RNase H Minus (Promega, Mannheim, Germany). Each RT-PCR reaction contained sense (0.5 μM) and antisense primer (0.5 μM) (table 1), 0.5 μl cDNA and GoTaq Polymerase (Promega, Mannheim, Germany). After 2 min at 95° C. cDNA was amplified (30 cycles) for 30 s at 95° C., 30 s at 57° C. and 1 min at 72° C. PCR products were visualized by loading on peqGREEN (Peqlab; Dusseldorf, Germany) containing agarose gels and analysed using ImageJ.

Western Blotting:

Protein was isolated from cells using a sample buffer containing 25 mM Tris-HCl, 150 mM NaCl, 100 mM dithiothreitol, 5.5% Nonidet P-40, 5% glycerol, 1 mM EDTA and 1% protease inhibitor mixture (Roche, cOmplete, EDTA-free, Mannheim, Germany). Proteins were separated by 7% sodium dodecyl sulfate (SDS) polyacrylamide gel and transferred to a polyvinylidene difluoride membrane (GE Healthcare Europe GmbH, Munich, Germany) or 4-20% Mini-PROTEAN TGX Stain-Free (Bio-Rad) using a semi-dry transfer unit (Bio-Rad). Membranes were incubated with primary anti-Tmem16a rabbit polyclonal antibody (Davids Biotech, Regensburg, Germany; 1:1000), anti-PKD1 (Polycystin-1 (7E12), Santa Cruz; 1:500) mouse antibody or anti-PKD2 (Polycystin-2 (D-3), Santa Cruz; 1:500) mouse antibody, overnight at 4° C. Proteins were visualized using horseradish peroxidase-conjugated secondary antibody and ECL detection. Actin was used as a loading control.

M1 Organoid Model:

M1 cells were resuspended as a single-cell suspension in 50/50% Matrigel/type I collagen and transferred into 24-well plates (30×10³ cells/well, four wells per condition) for 9 days. Medium was changed every 3 days. Every 3 days thirty random visual fields per well were photographed with an Axiovert 200 microscope (Zeiss, Germany). Cyst area of the lumina (˜30-150 cysts per condition and single experimental procedure) were measured with AxioVision (Zeiss, Germany). Cyst volume was then estimated using the formula for the volume of a sphere, 4/3πr³.

Immunocytochemistry:

M1 cells grown under confluent conditions for 4 days on glass coverslips and M1 organoids grown for 6 days were fixed for 10 min with methanol at −20° C. Organoids were isolated with ice cold 5 mM EDTA in PBS and seeded in poly-L-lysine coated coverslips. After seeded, cells were fixed for 10 min with methanol at −20° C. After washing, the cells were permeabilized with 0.5% (v/v, PBS) Triton X-100 for 10 min and blocked with 1% (w/v, PBS) bovine serum albumin for 1 h at room temperature. The cells were incubated overnight with primary antibodies (moo) against rabbit anti-TMEM16A (Davids Biotechnologie, Regensburg, Germany), or rat anti-Ki-67 (DAKO, M7249, Germany) or mouse anti-acetylated tubulin (T7451, Sigma-Aldrich, Germany). Binding of the primary antibody was visualized by incubation with appropriate secondary antibodies conjugated with Alexa Fluor 488 or Alexa Fluor 546 (1:300, Molecular Probes, Invitrogen). Nuclei were stained with Hoe33342 (0.1 g/ml PBS, AppliChem, Darmstadt, Germany). Glass coverslips were mounted on glass slides with fluorescent mounting medium (DakoCytomation, Hamburg, Germany) and examined with an ApoTome Axiovert 200M fluorescence microscope (Zeiss, Germany).

Cell Proliferation assay:

M1 cells were plated in 96-well plates at a density of 2×10³ cells per well for the time duration as indicated (0, 3, 6 and 9 days). Medium was changed every 3 days. Cells were incubated for 2 h in 100 μl of fresh media containing 0.5 mg/ml of the tetrazolium salt MTT. The dark blue formazan product was dissolved with DMSO and the absorbance measured at 595 nm.

Ussing Chamber:

MDCK or M1 cells were grown as polarized monolayers on permeable supports (Millipore MA, Germany) for 8 days. Cells were mounted into a perfused micro-Ussing chamber, and the luminal and basolateral surfaces of the epithelium were perfused continuously with Ringer's solution (mmol/l: NaCl 145; KH₂PO₄ 0.4; K₂HPO₄ 1.6; glucose 5; MgCl₂ 1; Ca²⁺ gluconate 1.3) at a rate of 5 ml/min (chamber volume 2 ml). Bath solutions were heated to 37° C., using a water jacket. Experiments were carried out under open circuit conditions. In addition, 100 μM ATP/UTP were added on the apical or basolateral side, or 100 μM 3-isobutyl-1-methylxanthine and 2 μM Forskolin (I/F) were added on the basolateral side, or 2 μM Ionomycin were added on the apical side, as indicated in the figure. Data were collected continuously using PowerLab (AD Instruments, Australia). Values for transepithelial voltages (Vₜₑ) were referred to the basolateral side of the epithelium. Transepithelial resistance (Rₜₑ) was determined by applying short (1 s) current pulses (ΔI=0.5 μA). Rₜₑ and equivalent short circuit currents (I_{′SC}) were calculated according to Ohm's law (Rₜₑ=ΔVₜₑ/ΔI, I_{′SC}=Vₜₑ/Rₜₑ).

Measurement of [Ca²⁺]i:

Primary cilium and membrane Ca²⁺ signals were detected after MDCK M2 and C7 cell were transfected with 5HT6-mCherry-GECO1.0 (5HT6-GECO, Addgene, Cambridge, Mass., USA). Cells were grown to confluence in glass coverslips and serum starved for 4-6 days to induce cilium formation. Afterwards, the cells were mounted and perfused in Ringer's solution. The mCherry fluorescence of the indicator was used to localize the Ca²⁺ sensor. Therefore, before each experiment, a photo was taken exciting the 5HT6-GECO at 560 nm, and the emission was recorded between 620±30 nm using a CCD-camera (CoolSnap HQ, Visitron Systems, Germany). To measure the ciliary Ca²⁺ changes, 5HT6-GECO was excited at 485/405 nm, and the emission was recorded between 535±12.5 nm. The results for [Ca²⁺ ]cilium and [Ca²⁺]cyt were obtained at 485/405 nm changes and given in ratio. Measurement of the global cytosolic Ca²⁺ changes were performed as described recently. In brief, cells were loaded with 5 μM Fura-2, AM (Molecular Probes) in OptiMEM (Invitogen) with 0.02% pluronic (Molecular Probes) for 1 h at RT and 30 min at 37° C. Fura-2 was excited at 340/380 nm, and the emission was recorded between 470 and 550 nm using a CCD-camera (CoolSnap HQ, Visitron Systems, Germany). Control of experiment, imaging acquisition, and data analysis were done with the software package Meta-Fluor (Universal imaging, USA). [Ca2+]ᵢ was calculated from the 340/380 nm fluorescence ratio after background subtraction. The formula used to calculate [Ca²⁺]ᵢ was [Ca^{2+]}ᵢ=Kd×(R−Rₘᵢₙ)/(Rₘₐₓ−R)×(S_{f2}/S_{b2}), where R is the observed fluorescence ratio. The values Rₘₐₓ and Rₘᵢₙ (maximum and minimum ratios) and the constant S_{f2/}S_{b2} (fluorescence of free and Ca²⁺-bound Fura-2 at 380 nm) were calculated using 1 μmol/liter ionomycin (Calbiochem), 5 μmol/liter nigericin, 10 μmol/liter monensin (Sigma), and 5 mmol/liter EGTA to equilibrate intracellular and extracellular Ca²⁺ in intact Fura-2-loaded cells. The dissociation constant for the Fura-2·Ca²⁺ complex was taken as 224 nmol/liter. ER Ca²⁺ signals were detected in Ca²⁺ sensor ER-LAR-GECO1 (Addgene, Cambridge, Mass., USA) expressing M1 cells. Cells were excited at 560 nm and emission was recorded between 620±30 nm.

Materials and Statistical Analysis:

All compounds used were of highest available grade of purity. Data are reported as mean±SEM. Student's t-test for unpaired samples and ANOVA were used for statistical analysis. p<0.05 was accepted as significant difference.

### Example 2

In the following, results are presented showing the importance of TMEM16A in polycystic kidney disease and/or polycystic liver disease, as well as the potential of TMEM16A inhibitors benzbromarone and/or niclosamide for the treatment of PKD/PLD.

TMEM16A Augments Fluid Secretion by Increase in Intracellular Ca²⁺:

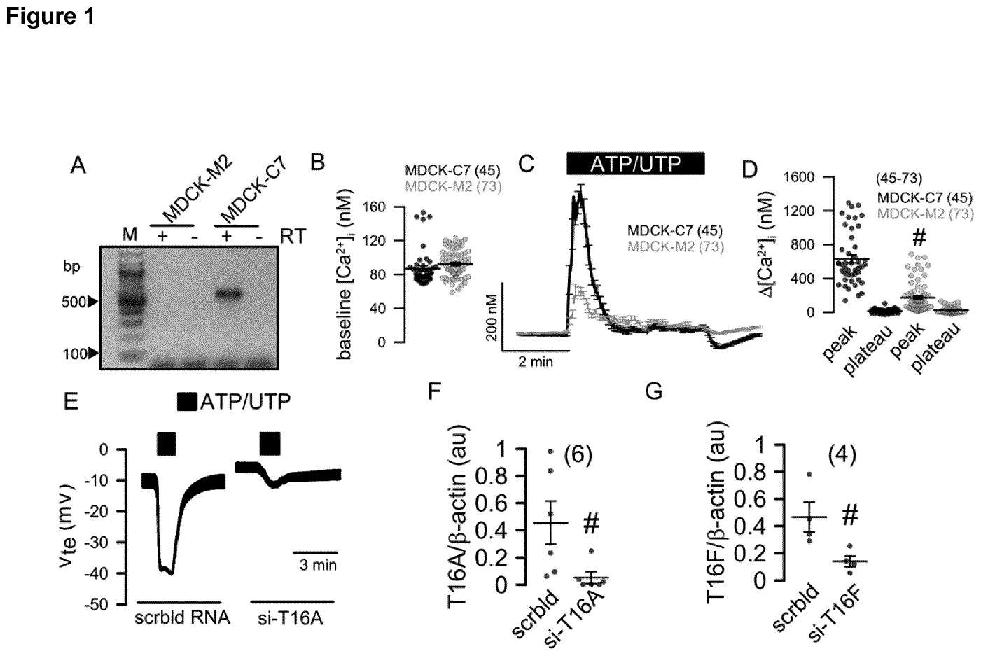

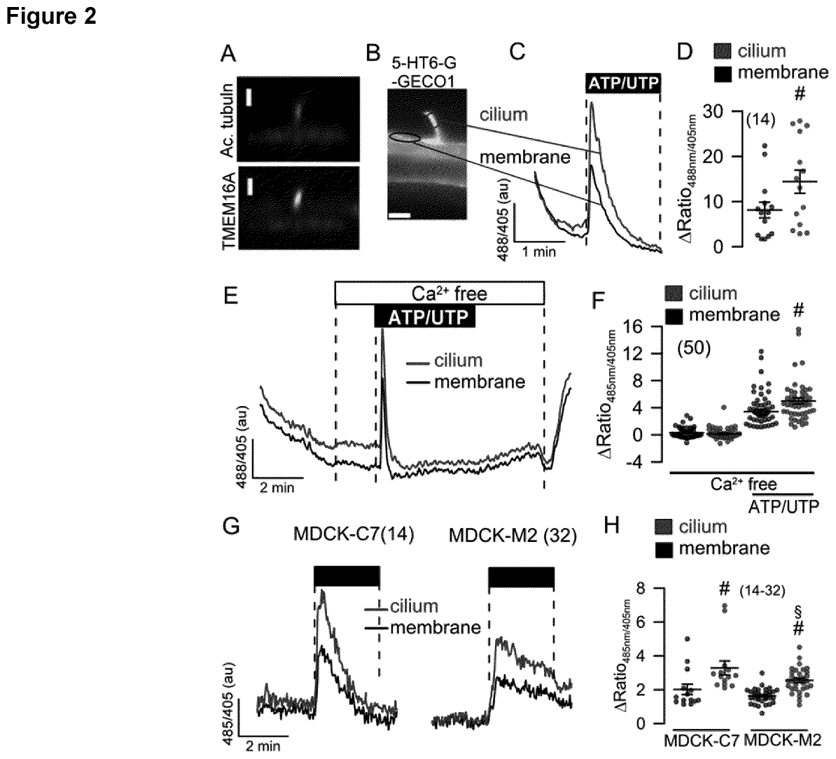

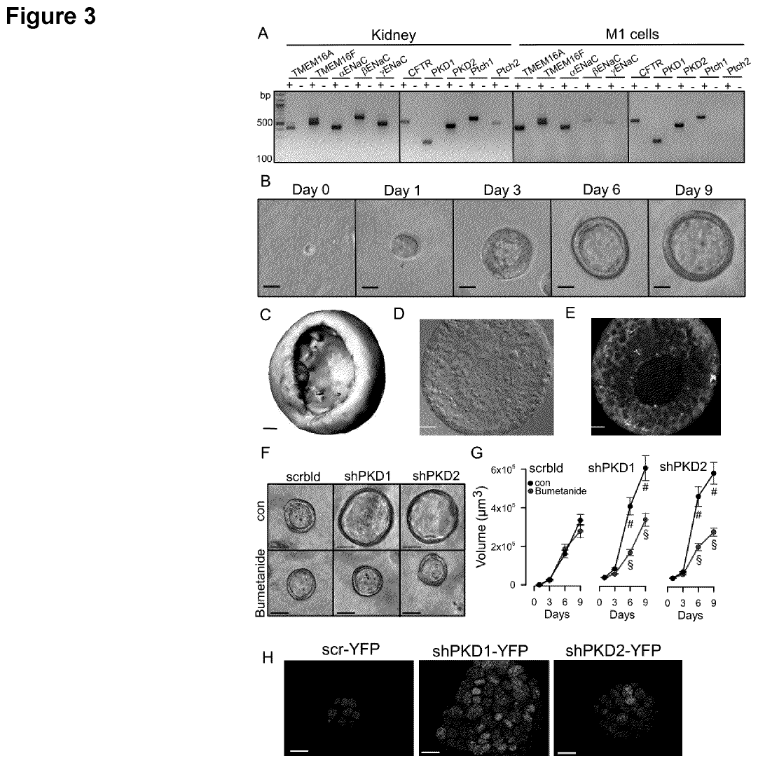

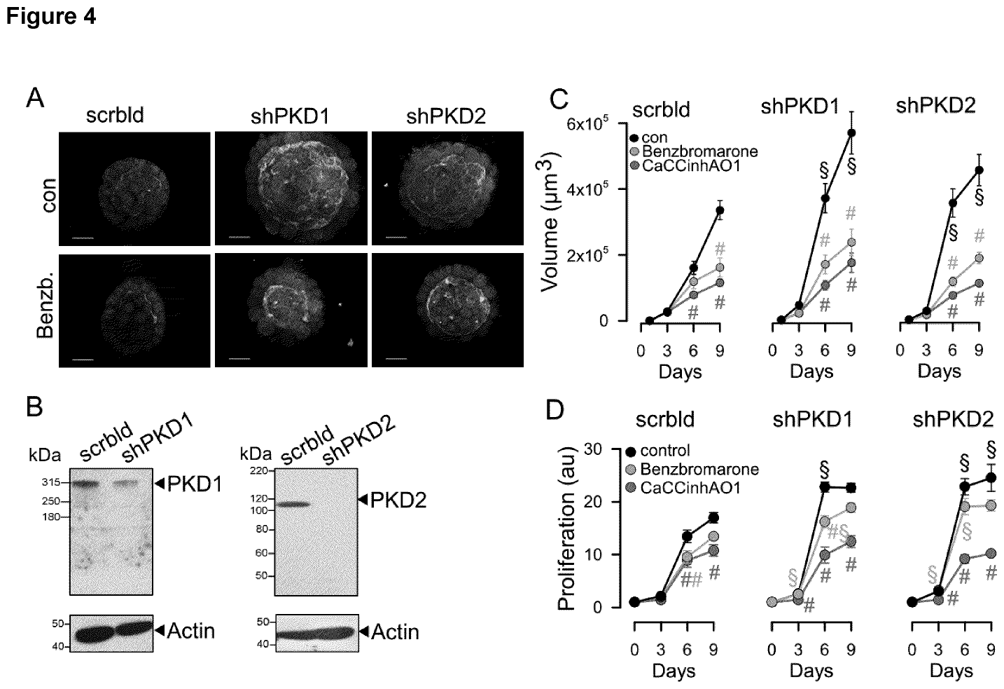

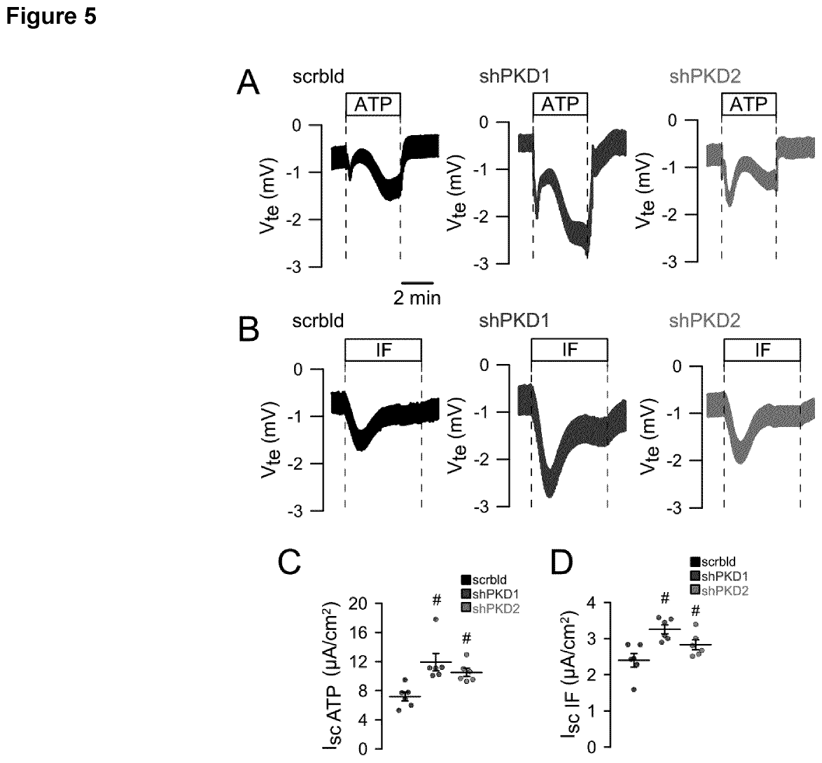

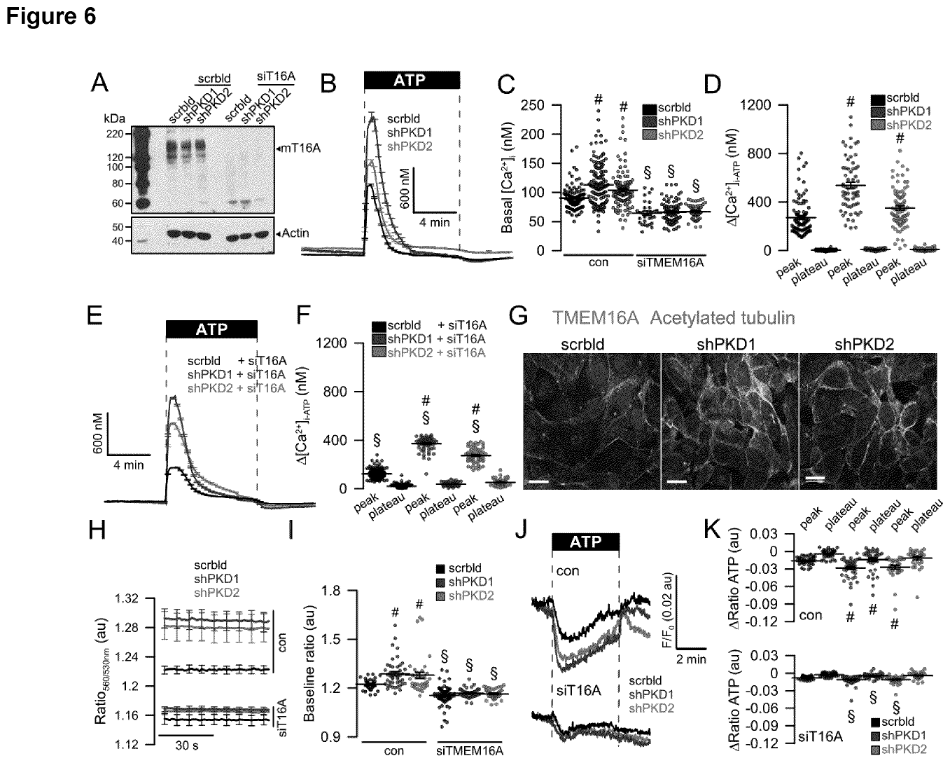

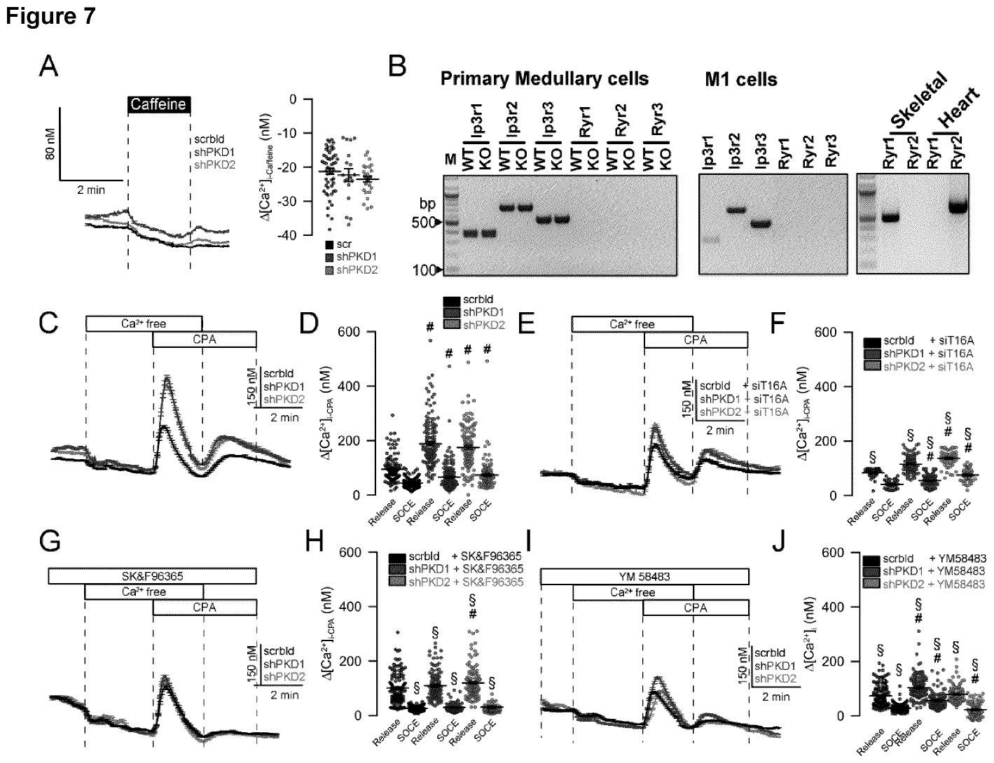

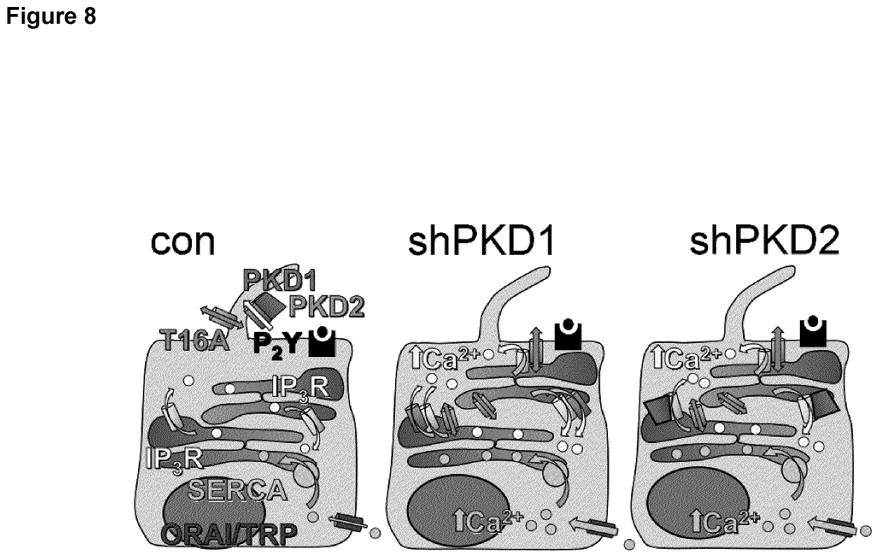

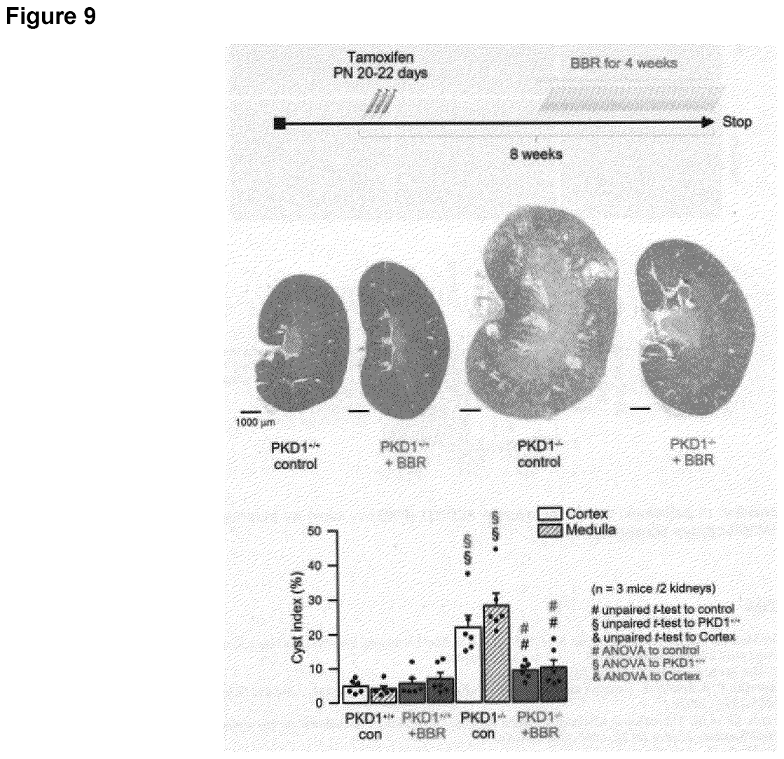

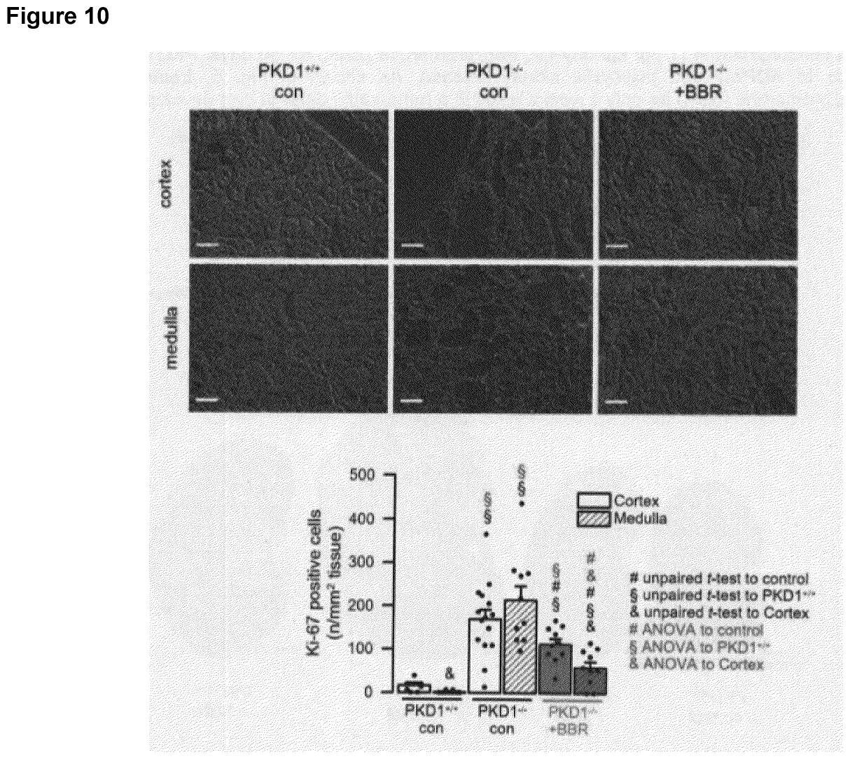

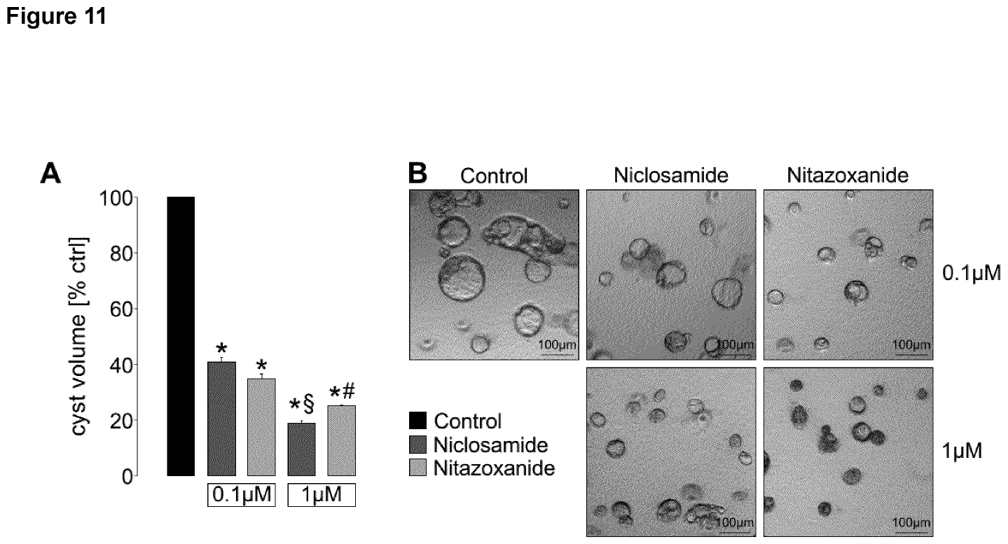

The impact of TMEM16A on fluid secretion and cyst growth in a MDCK cyst model and in embryonic kidney cultures was described previously by the present inventors. MDCK cells derived from dog principal cells exist as a TMEM16A-expressing MDCK-C7 clone and as a MDCK-M2 clone, which lacks expression of TMEM16A (FIG. 1A). C7 cells show a remarkable increase in intracellular Ca²⁺ and a pronounced Cl⁻ secretion when stimulated with the purinergic receptor agonists ATP (100 μM) or UTP (100 μM) (FIG. 1B-D). SiRNA-knockout of TMEM16A inhibited Cl⁻ secretion by purinergic receptor stimulation (FIG. 1E). Furthermore, siRNA-knockdown of TMEM16F did not affect Ca²⁺ activated Cl⁻ currents (data not shown; FIG. 1F,G). TMEM16A is expressed in plasma membrane and primary cilium (FIG. 2A). Ca²⁺ changes in primary cilium and near the plasma membrane were measured using 5-HT6-G-GECO1 (FIG. 2B). A Ca²⁺ rise in both cilium and near plasma membrane was detected upon purinergic receptor stimulation with ATP or UTP (FIG. 2C,D). Purinergic Ca²⁺ rise was larger in the primary cilium than close to the plasma membrane, but otherwise qualitatively similar. It was attenuated in MDCK-M2 cells lacking expression of TMEM16A (FIG. 2G,H).

Loss of PKD1 or PKD2 Induces Cl⁻ Secretion in M1 Renal Organoids:

The present inventors examined the role of TMEM16A for Ca²⁺ signaling and renal cyst growth, as well as the impact of polycystins in an improved M1 mouse collecting duct model. M1 cells show expression of polycystins (PKD1, PKD2), TMEM16A, TMEM16F, CFTR, and ENaC subunits similar to native mouse medullary kidney cells (FIG. 3A). M1 cells readily produce spherical renal organoids when grown as a 3D culture in matrigel (FIG. 3B,C). The cells appear highly differentiated and form primary cilia (FIG. 3D,E). Importantly, M1 renal organoids do not seem to secrete fluid, because the NKCC1 inhibitor bumetanide did not interfere with the formation of the organoid (FIG. 3F,G). However, they express epithelial Na⁺ channels and increase their volume when grown in amiloride (not shown). In contrast, knockdown of either PKD1 or PKD2 increased the organoid volume, and this increase in volume was inhibited by bumetanide, indicating activation of ion secretion upon knockdown of polycystins and induction of a cystic phenotype (FIG. 3F,G, FIG. 4C).

In a renal organoid model with M1 collecting duct cells, the present inventors found upregulation of TMEM16A with loss of expression of PKD1 or PKD2. TMEM16A supports Ca²⁺ store release, cell proliferation and fluid secretion and thereby contributes to cyst growth. TMEM16A therefore contributes to the pathogenic events observed in ADPKD.

Enhanced Secretion and Proliferation in PKD Requires TMEM16A:

A hallmark of renal cysts is the upregulation of proliferation. Ki-67 staining in M1 renal organoids caused strong upregulation of proliferation upon knockdown of PKD1 or PKD2 (FIG. 3H). Notably with knockdown of PKD1 or PKD2 and increase in proliferation, expression of TMEM16A was strongly increased (FIG. 4A). Benzbromarone or CaCCinhAO1, two potent inhibitors of TMEM16A, blocked increase in volume and proliferation (FIG. 4A-D). When grown as 2D cultures on permeable supports, cells with knockdown of PKD1 or PKD2 demonstrated larger ATP-activated TMEM16A and cAMP-activated CFTR currents (FIG. 5). The data suggest that enhanced secretion and proliferation caused by knockdown of PKD1 or PKD2 is strongly dependent on TMEM16A.

Disturbed Ca²⁺ Signaling in PKD Relies on TMEM16A:

Abrogated Ca²⁺ signaling in ADPKD has been intensely examined, but controversial results have been reported. The present inventors reported a role of TMEM16A in Ca²⁺ signaling, i.e. enhanced agonist-induced Ca²⁺-store release by TMEM16A. Herein the present inventors show the impact of TMEM16A on ER Ca²⁺-store release through IP₃R and ryanodine receptors (RyR) upon knockdown of PKD1 and PKD2 (FIG. 6A). Knockdown of PKD1 or PKD2 enhanced basal [Ca²⁺], and augmented ATP-induced store release (FIG. 6B-D). The enhanced Ca²⁺ signals observed in the absence of PKD1 or PKD2 required the presence of TMEM16A, as both basal Ca²⁺ levels and ATP-induced store release were strongly attenuated by knockdown of TMEM16A (FIG. 6C-F). Similar to M1-organoids, also M1-monolayers demonstrated lower expression levels for TMEM16A when compared to M1 cells with knockout in PKD1 or PKD2 (FIG. 6G). Using the ER Ca²⁺ sensor ER-LAR-GECO1, the present inventors found higher basal ER Ca²⁺ levels and enhanced ATP-induced Ca²⁺ release in cells lacking expression of PKD1 or PKD2 (FIG. 6H-J). In contrast, knockdown of TMEM16A strongly reduced store filling and ATP-induced Ca²⁺-release (FIG. 6K).

Upregulated TMEIM6A Causes Enhanced ER Store Release and Store Refill in ADPKD:

Ryanodine receptors (RyR) are inhibited by Polycystin-2 in mouse heart and have been reported to operate as Ca²⁺ release channels in cultured human renal epithelial cells. RyR was reported to have an essential role in flow-induced Ca²⁺ increase in mouse kidney. However, the activator of RyR, caffeine, did not increase intracellular Ca²⁺, and the present inventors did not detect expression of RyR1-3 in mouse wt and PKD1−/− primary renal epithelial and M1 collecting duct cells (FIG. 7A,B). In contrast, signals for RyR1-3 were clearly present in skeletal muscle, heart muscle, and brain, respectively (not shown). The present inventors therefore conclude that RyR are not relevant for changes in Ca²⁺ signaling induced by knockout of polycystins in mouse renal epithelial cells. Lack of PKD1 or PKD2 increased store emptying induced by inhibition of SERCA with cyclopiazonic acid (CPA). Moreover, store operated Ca²⁺ entry (SOCE) was also enhanced by knockdown of PKD1/PKD2 (FIG. 7C,D). Enhanced store release and enhanced SOCE was strongly reduced in the absence of TMEM16A (FIG. 7E,F). Moreover, the inhibitor of transient receptor potential (TRP) channels SK&F96365 and the ORAL inhibitor YM58483 inhibited enhanced Ca²⁺ entry in PKD1/PKD2 knockout cells and abolished enhanced CPA-induced store release (FIG. 7G-J). Taken together the present data demonstrate augmented Ca²⁺ signals in the absence of either PKD1 or PKD2. Enhanced Ca²⁺ signaling requires the presence of the TMEM16A Cl⁻ channel, which therefore represents a suitable drug target in ADPKD (FIG. 8).

### Example 3

Aberrant intracellular Ca²⁺ signaling, enhanced cell proliferation and fluid secretion are essential factors that drive growth of renal cysts. The present inventors herein demonstrate ATP-induced Ca²⁺ increase in both the primary cilium as well as in the cytosol near the plasma membrane of MDCK cells (FIG. 2). Although ciliary Ca²⁺ increase by ATP was larger, the responses in the cilium and cytoplasm were similar. The present inventors therefore continued to analyze cytosolic Ca²⁺ changes.

Inhibition of the IP₃ receptors by PKD1 with attenuation of Ca²⁺ release from IP₃-sensitive stores has been reported earlier. Accordingly, receptor mediated Ca²⁺ release is enhanced with the loss of PKD1. The present inventors show that a lack of PKD1 is likely to augment store operated calcium entry, which was detected in the herein disclosed study (FIG. 7). Enhanced Ca²⁺ entry was blocked by the inhibitor of receptor-mediated Ca²⁺ entry SK&F96365, and by the inhibitor of store operated ORAI1 Ca²⁺ influx channels, YM58483 (FIG. 7). Enhanced (and mislocalized) expression of PKD2 in the ER in the absence of PKD1 is likely to operate as a Ca²⁺ activated ER Ca²⁺ leakage channel, which will contribute to enhanced Ca²⁺ release from IP₃-sensitive (IP₃R) stores (FIG. 8). Notably, abnormal Ca²⁺ permeability of the ER membrane in ADPKD may account for both change in apoptotic activity and increased proliferation.

TMEM16A channels enhance ER-Ca²⁺ store release by sequestering the ER and IP3 receptors to Ca²⁺ signaling compartments near the plasma membrane. ER-located TMEM16A supports both release of Ca²⁺ from intracellular ER-Ca²⁺ stores, as well as reuptake of Ca²⁺ by the SERCA (FIG. 8). In contrast to earlier reports, the present inventors did not detect expression of RyR channels or effects of caffeine on [Ca²⁺]ᵢ in mouse primary renal epithelial cells or M1 cells (FIG. 7I,J).

The expression of TMEM16A being upregulated through activation of STATE (and STAT3) may be the reasons for the upregulation of TMEM16A in M1 cysts observed in the study disclosed herein (FIG. 4A). TMEM16A supports proliferation, cell migration and development of cancer by recruiting a number of intracellular signaling pathways. Conclusively, the present inventors show herein that TMEM16A is a highly potential drug target for treating polycystic kidney disease.

### Example 4

Inhibition of Cyst Growth In Vivo

Inducible and tubule-specific PKD1 knockout (PKD1−/−) leads to ADPKD and polycystic kidney disease. Wt mice and mice with a knockout in the gene PKD1 were treated with benzbromarone (1 μg/kg/day intraperitoneal (I.P.) benzbromarone (BBR)) for 30 days starting 4 weeks after induction of the PKD1 knockout at postnatal (PN) 20-22. As shown in FIG. 9, treatment with benzbromarone (BBR) for only 4 weeks leads to a remarkable delay in cyst development.

Cell proliferation was examined in kidneys of control animals (PKD+/+) and PKD1−/− animals using the proliferation marker Ki-67. As shown in FIG. 10, the treatment with the TMEM16A-inhibitor benzbromarone largely abolished pathologic proliferation in ADPKD (PKD1−/− mice).

### Example 5

Polycystic kidney disease (PKD) leads to continuous decline of renal function by growth of renal cysts. Enhanced proliferation and transepithelial chloride secretion through cystic fibrosis transmembrane conductance regulator (CFTR) is observed to cause an increase in cyst volume. Ca²⁺ activated Cl⁻ channel TMEM16A (anoctamin 1) has a pro-proliferative role and TMEM16A contributes to CFTR-dependent Cl⁻ secretion. The present application demonstrates an increase in intracellular Ca²⁺ ([Ca²⁺]i) signals and Cl⁻ secretion by TMEM16A, in renal collecting duct principal cells from dog (MDCK) and mouse (M1). M1 organoids strongly proliferate, increase expression of TMEM16A and secrete Cl⁻ upon knockdown of endogenous polycystin-1 or -2 (PKD1,2) by retroviral transfection of shRNA directed against PKD1 and PKD2 (shPKD1 and shPKD2), respectively. Knockdown of PKD1 or PKD2 increased basal intracellular Ca²⁺ levels and enhanced purinergic/inositol trisphosphate (IP3)-induced Ca²⁺ release from endoplasmic reticulum. In contrast, ryanodine receptors were not expressed and caffeine had no effects on [Ca²⁺]i. Ca²⁺ signals, proliferation and Cl⁻ secretion were largely reduced by knockdown or blockade of TMEM16A. Thus, the present inventors conclude that TMEM16A is essential for enhanced Ca²⁺ release from IP3-sensitive Ca²⁺ stores in autosomal dominant polycystic kidney disease (ADPKD). The data suggest TMEM16A as a major pathogenic factor during ADPKD, and thus represents a suitable therapeutic target in polycystic kidney disease.

### Example 6

The effect of niclosamide and derivatives thereof, e.g. nitazoxanide, on cyst growth was analyzed. Particularly, it was analyzed whether there is a dose-dependent effect on cyst growth. Polycystin-1-deficient collecting duct (p1MDCK) cells were resuspended in a collagen I matrix to form cysts in vitro and were cultured in the presence of lovIM forskolin for 5 days. The cells were treated with either 0.1 μM or 1 μM niclosamide, or with 0.1 μM or 1 μM of an exemplary niclosamide derivative, namely nitazoxanide. It was shown (FIG. 11) that the mean cyst volumes were significantly decreased when treated with niclosamide or niclosamide derivative nitazoxanide. Niclosamide and derivative nitazoxanide efficiently inhibit cyst growth in vitro. Furthermore, higher concentrations of niclosamide and niclosamide derivative, namely 1 μM, achieved a higher reduction in cyst volumes than lower concentrations, namely 0.1 μM, of niclosamide and niclosamide derivative. Conclusively, both niclosamide and niclosamide derivative nitazoxanide effectively inhibit cyst growth.

### REFERENCES

[1] Huang, F. et al. Calcium-activated chloride channel TMEM16A modulates mucin secretion and airway smooth muscle contraction. Proc. Natl. Acad. Sci U.S.A 109, 16354-16359 (2012).
[2] Miner, K. et al. The Anthelminthic Niclosamide And Related Compounds Represent Potent Tmem16a Antagonists That Fully Relax Mouse And Human Airway Rings. Frontiers in pharmacology 14,10:51 (2019).
[3] Schreiber et al. Lipid peroxidation drives renal cyst growth in vitro through activation of TMEM16A. J. Am. Soc. Nephrol. 30: 228-242 (2019).
[4] Buchholz B, et al. Anoctamin 1 induces calcium-activated chloride secretion and proliferation of renal cyst-forming epithelial cells. Kidney international advance online publication, 23 Oct. 2013.
[5] Irazabal M V, Rangel L J, Bergstralh E J et al. Imaging Classification of Autosomal Dominant Polycystic Kidney Disease: A Simple Model for Selecting Patients for Clinical Trials. J. Am. Soc. Nephrol. (2015) 26: 160-172.
[6] Gansevoort R T, Arici M, Benzing T et al. Recommendations for the use of tolvaptan in autosomal dominant polycystic kidney disease: a position statement on behalf of the ERA-EDTA Working Groups on Inherited Kidney Disorders and European Renal Best Practice. Nephrol. Dial. Transpl. (2016) 31: 337-48.

The features of the present invention disclosed in the specification, the claims, and/or in the accompanying figures may, both separately and in any combination thereof, be material for realizing the invention in various forms thereof.

1. A method of treating and/or preventing a pathological condition selected from polycystic kidney disease, polycystic liver disease, and a combination thereof, wherein said method comprises administering a compound which is a TMEM16 inhibitor selected from benzbromarone, niclosamide, and pharmaceutically acceptable salts thereof, to a patient in need thereof.

2. The method according to claim 1, wherein said pathological condition is a combination of polycystic kidney disease and polycystic liver disease.

3. The method according to claim 1, wherein said pathological condition is characterized by cyst development.

4. The method according to claim 1, wherein said pathological condition is characterized by increased TMEM16A expression and/or increased TMEM16F expression.

5. The method according to claim 1, wherein said polycystic kidney disease is autosomal dominant polycystic kidney disease (ADPKD) or autosomal recessive polycystic kidney disease (ARPKD).

6. The method according to claim 1, wherein said compound is capable of inhibiting renal cyst growth and/or hepatic cyst growth by inhibiting TMEM16A and/or TMEM16F.

7. The method according to claim 1, wherein said compound is administered in an amount of from 10 mg per day to 800 mg per day.

8. The method according to claim 1, wherein said compound is administered once every 4-8 h, once daily, or once weekly.

9. The method according to claim 1, wherein said compound is administered to a patient in need thereof, wherein said patient is a mammal, preferably a human.

10. The method according to claim 1, wherein said compound is administered topically or systemically.

11. The method according to claim 1, wherein said compound is administered intravenously, intravascularly, orally, intraarticularly, nasally, mucosally, intrabronchially, intrapulmonarily, intrarenally, intrahepatically, intradermally, subcutaneously, intramuscularly, intraocularly, intrathecally, or intranodally.

12. The method according to claim 1, wherein said compound is co-administered with an agent selected from an antihypertensive agent, an antiinfective agent, an antibiotic agent, an analgesic agent, a vasopressin antagonist such as tolvaptan, a somatostatin analogue such as octreotide, and an mTOR antagonist such as sirolimus or everolimus.

13. The method according to claim 1, wherein said compound is a biologically active derivative of benzbromarone or a biologically active derivative of niclosamide.

14. The method according to claim 1, wherein said compound is administered as a composition wherein said composition comprises said compound and a pharmaceutically acceptable excipient.

15. The method according to claim 14, wherein said composition further comprises any of an antihypertensive agent, an antiinfective agent, an antibiotic agent, an analgesic agent, a vasopressin antagonist such as tolvaptan, a somatostatin analogue such as octreotide, an mTOR antagonist such as sirolimus or everolimus, a disintegrant, and a pharmaceutically acceptable carrier.

16. The method according to claim 4, wherein said pathological condition is characterized by increased TMEM16A expression in kidney cells.

17. The method according to claim 5, wherein said polycystic kidney disease is ADPKD.

18. The method according to claim 7, wherein said compound is administered in an amount of from 40 mg to 600 mg per day.

19. The method according to claim 8, wherein said compound is administered once daily.

20. The method according to claim 9, wherein said patient is a human.

21. The method according to claim 11, wherein said compound is administered orally.
